(19) United States
(12) Patent Application Publication
(10) Pub. No.: US 2023/0001158 A1
(43) Pub. Date: Jan. 5, 2023

(54) Instrument Delivery Device With Different Guidewire and Tubing Advancement Ratios

(71) Applicant: Becton, Dickinson and Company, Franklin Lakes, NJ (US)

(72) Inventors: Jonathan Karl Burkholz, Salt Lake City, UT (US); Yiping Ma, Layton, UT (US)

(21) Appl. No.: 17/851,621

(22) Filed: Jun. 28, 2022

Related U.S. Application Data
(60) Provisional application No. 63/218,028, filed on Jul. 2, 2021.

(51) Int. Cl. A61M 25/09; A61B 5/15; A61M 39/10

(52) U.S. Cl. CPC A61M 25/09; A61B 5/150992; A61B 5/15003; A61M 39/10

(57) ABSTRACT

An instrument delivery device may include a housing, which may include a proximal end, a distal end, and a slot. The instrument delivery device may include an advancement element extending through the slot and configured to move linearly along the slot. The instrument delivery device may include a guidewire, which may include a first end and a second end. In response to movement of the advancement element distally a first distance along the slot, the second end of the guidewire may be configured to advance a second distance. The second distance may be at least twice the first distance. The instrument delivery device may include a tubing, which may include a distal end and a proximal end. In response to movement of the advancement element distally the first distance along the slot, the proximal end of the tubing may be configured to advance the first distance.

### CROSS-REFERENCE TO RELATED APPLICATION

The present application claims priority to U.S. Provisional Application Ser. No. 62/218,028, entitled “Instrument Delivery Device with Different Guidewire and Tubing Advancement Ratios”, filed Jul. 2, 2021, the entire disclosure of which is hereby incorporated by reference in its' entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increase. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related systems and methods. In particular, the present disclosure relates to instrument delivery devices configured to deliver one or more instruments into a catheter assembly and/or beyond the catheter assembly into vasculature of a patient to bypass or hold a fibrin sheath or thrombus away from the catheter opening and improve blood flow into the catheter assembly. In some embodiments, the instruments may include a tubing and/or a guidewire.

In some embodiments, an instrument delivery device may include a housing, which may include a proximal end, a distal end, and a slot. In some embodiments, the instrument delivery device may include an advancement element, which may extend through the slot and be configured to move linearly along the slot. In some embodiments, the instrument delivery device may include a guidewire, which may include a first end and a second end.

In some embodiments, in response to movement of the advancement element distally a first distance along the slot, the second end of the guidewire may be configured to advance a second distance. In some embodiments, the second distance may be twice the first distance. In these embodiments, the advancement element and the guidewire may have a 1:2 advancement ratio such that for a particular distance the advancement element is moved along the slot, the second end of the guidewire is moved twice the particular distance.

In some embodiments, the instrument delivery device may include a tubing, which may include a distal end and a proximal end. In some embodiments, in response to movement of the advancement element distally the first distance along the slot, the distal end of the tubing is configured to advance the first distance. In these embodiments, the advancement element and the tubing may have a 1:1 advancement ratio such that for a particular distance the advancement element is moved along the slot, the distal end of the tubing is moved the particular distance.

In some embodiments, the proximal end of the tubing may be coupled to the advancement element. In some embodiments, the instrument delivery device may include an extension tube coupled to the advancement element, and a blood collection pathway may extend through the tubing, the advancement element, and the extension tube.

In some embodiments, the instrument delivery device may include a seal disposed within the advancement element and preventing fluid communication between the blood collection pathway and a portion of a guidewire pathway. In some embodiments, the advancement element may include an arc-shaped channel, and the guidewire may be configured to move within the arc-shaped channel. In some embodiments, the first end of the guidewire may be secured within the housing.

In some embodiments, in response to movement of the advancement element distally the first distance along the slot, the second end of the guidewire and the distal end of the tubing may move from inside the housing to outside of the housing. In some embodiments, a distal end of the housing may include a distal connector, and in response to the advancement element being disposed at a proximal end of the slot, the second end of the guidewire and the distal end of the tubing may be aligned with a distal end of the distal connector. In some embodiments, the distal end of the housing may include the distal connector, and in response to the advancement element being disposed at a proximal end of the slot, the second end of the guidewire and the distal end of the tubing may be proximal to the distal end of the distal connector.

In some embodiments, an inner surface of the housing may include a groove disposed between the proximal end of the housing and the distal end of the housing. In some embodiments, the guidewire and/or the tubing may be disposed within the groove.

In some embodiments, an instrument delivery device may include a housing, which may include a proximal end, a distal end, and a slot. In some embodiments, the instrument delivery device may include a first advancement element extending through the slot and configured to move linearly along the slot. In some embodiments, the instrument delivery device may include a second advancement element extending through the slot and configured to move linearly along the slot.

In some embodiments, the instrument delivery device may include a guidewire, which may include a first end and a second end. In some embodiments, in response to movement of the first advancement element distally a first distance along the slot, the second end of the guidewire may be configured to advance a second distance. In some embodiments, the second distance may be twice the first distance. In these embodiments, the first advancement element and the guidewire may have a 1:2 advancement ratio such that for a particular distance the first advancement element is moved along the slot, the second end of the guidewire is moved twice the particular distance.

In some embodiments, the instrument delivery device may include a tubing, which may include a distal end and a proximal end. In response to movement of the second advancement element distally the first distance along the slot, the distal end of the tubing may be configured to advance the first distance. In these embodiments, the second advancement element and the tubing may have a 1:1 advancement ratio such that for a particular distance the second advancement element is moved along the slot, the distal end of the tubing is moved the particular distance.

In some embodiments, the proximal end of the tubing may be coupled to the second advancement element. In some embodiments, the instrument delivery device may include another tubing coupled to the second advancement element. In some embodiments, a blood collection pathway may extend through the tubing, the second advancement element, and the other tubing. In some embodiments, the instrument delivery device may include a seal disposed within the second advancement element and preventing fluid communication between the blood collection pathway and a portion of a guidewire pathway. In some embodiments, the guidewire may extend through the seal.

In some embodiments, the first advancement element may include an arc-shaped channel, and the guidewire may move within the arc-shaped channel. In some embodiments, the first end of the guidewire may be secured within the housing. In some embodiments, the second advancement element may be distal to the first advancement element. In some embodiments, in response to movement of the first advancement element distally the first distance along the slot, the second end of the guidewire and the distal end of the tubing may move from inside the housing to outside of the housing.

In some embodiments, an instrument delivery device may include a housing, which may include a distal end, a proximal end, and a slot disposed between the distal end and the proximal end. In some embodiments, the distal end may be configured to couple to an intravenous catheter device. In some embodiments, the instrument delivery device may include a tubing, which may include a distal end and a proximal end.

In some embodiments, the instrument delivery device may include an advancement element through the slot and configured to move linearly along the slot. In some embodiments, the advancement element may be coupled to the proximal end of the tubing. In some embodiments, in response to movement of the advancement element distally a first distance along the slot, the distal end of the tubing may be configured to advance the first distance.

In some embodiments, the instrument delivery device may include an instrument disposed within the housing. In some embodiments, the instrument delivery device may include an advancement wheel. In some embodiments, the advancement wheel may extend out from the housing. In some embodiments, in response to the advancement wheel being rotated, the instrument may be configured to advance through the advancement element, the tubing, and the distal end of the housing. In some embodiments, the instrument may include a guidewire.

In some embodiments, the instruments delivery device may include a septum disposed within the advancement element and configured to prevent blood from flowing into the housing. In some embodiments, the guidewire may be configured to move through the septum. In some embodiments, the instrument delivery device may include another tubing coupled to the advancement element and configured to couple to a blood collection device. In some embodiments, blood may be configured to flow proximally through the tubing, the advancement element, and the other tubing.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example instrument delivery device, illustrating an example advancement element in an example initial or retracted position, according to some embodiments;

FIG. 1B is a cross-sectional view of the instrument delivery device of FIG. 1A;

FIG. 1C is an enlarged cross-sectional view of a portion of the instrument delivery device of FIG. 1A, according to some embodiments;

FIG. 1D is a cross-sectional view of the instrument delivery device of FIG. 1A along the line 1D-1D of FIG. 1A, according to some embodiments;

FIG. 1E is an enlarged view of a portion of FIG. 1D, according to some embodiments;

FIG. 2A is an upper perspective view of the instrument delivery device of FIG. 1A coupled to an example catheter assembly, illustrating the advancement element in an example fully advanced position, according to some embodiments;

FIG. 2B is an upper perspective view of a distal end of the instrument delivery device of FIG. 1A when the advancement element is in an example fully advanced position, according to some embodiments;

FIG. 2C is a schematic diagram of the instrument delivery device of FIG. 1A in an example initial or retracted position, an example partially advanced position, and an example fully advanced position, according to some embodiments;

FIG. 3A is a cross-sectional view of another instrument delivery device, illustrating the advancement element in an example initial or retracted position, according to some embodiments;

FIG. 3B is a schematic diagram of the instrument delivery device of FIG. 3A in an example initial or retracted position, an example partially advanced position, and an example fully advanced position, according to some embodiments;

FIG. 4A is a cross-sectional view of another instrument delivery device, illustrating an example first advancement element in an example initial or retracted position, according to some embodiments;

FIG. 4B is a schematic diagram of the instrument delivery device of FIG. 4A, according to some embodiments;

FIG. 5A is a cross-sectional view of another instrument delivery device, illustrating an example guidewire and an example tubing in an example initial or retracted position, according to some embodiments; and

FIG. 5B is an exploded rear view of an example instrument delivery mechanism of the instrument delivery device of FIG. 5A, according to some embodiments.

### DETAILED DESCRIPTION

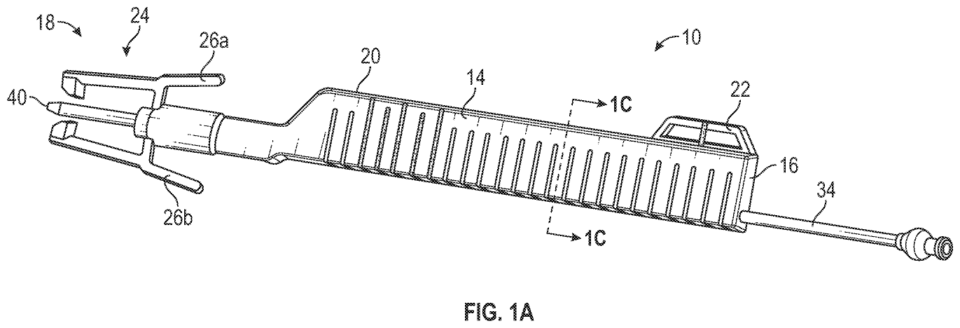

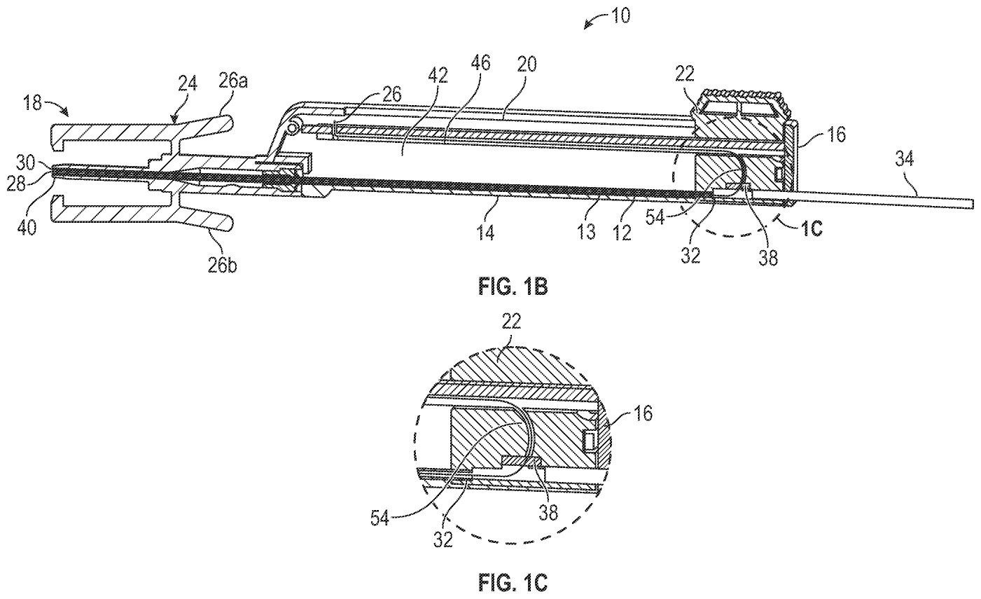

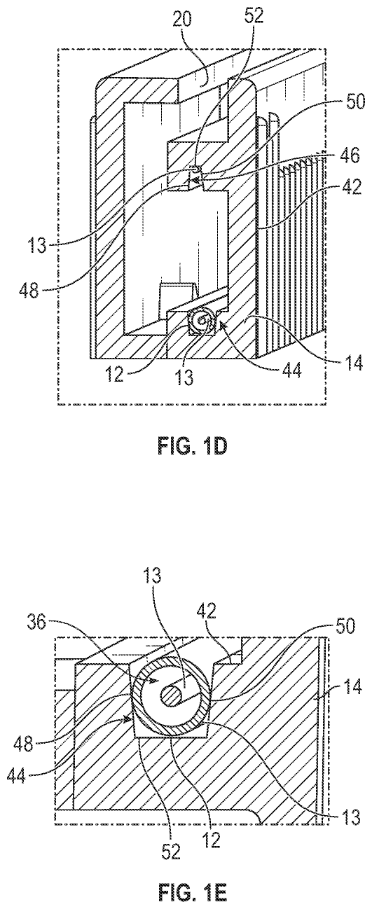

Referring now to FIGS. 1A-1E, in some embodiments, an instrument delivery device 10 may be configured to deliver a tubing 12 and/or a guidewire 13 into or through a catheter assembly. In some embodiments, the instrument delivery device 10 may provide needle-free delivery of the tubing 12 and/or the guidewire 13 into or through the catheter assembly. In some embodiments, the tubing 12 and/or the guidewire 13 may be advanced through a catheter of the catheter assembly to push past any occlusions in the catheter or vasculature (e.g., a thrombus or a fibrin sheath at a tip of the catheter, vein collapse, valves, etc.) to create a clear pathway for fluid flow into the catheter assembly, which may aid in blood collection. In some embodiments, the tubing 12 and/or a guidewire 13 may reduce or remove occlusions, improving patency of the catheter for medication and fluid delivery, as well as blood acquisition during a dwell time of the catheter. In some embodiments, the instrument delivery device 10 may improve a blood collection flow rate, blood sample quality, and fluid path robustness, while maintaining a small size to facilitate handling by a user.

In some embodiments, the catheter may include a peripheral intravenous (IV) catheter, a peripherally-inserted central catheter, or a midline catheter. In some embodiments, the catheter through which the tubing 12 and/or the guidewire 13 are delivered may have been previously inserted into vasculature of a patient and may be dwelling within the vasculature when the tubing 12 and/or the guidewire 13 is advanced into the catheter assembly.

In some embodiments, the tubing 12 and/or the guidewire 13 may be disposed within a housing 14, which may be configured to protect the tubing 12 from damage and/or contamination from a surrounding external environment. In some embodiments, the housing 14 may be rigid or semi-rigid. In some embodiments, the housing 14 may be made of one or more of stainless steel, aluminum, polycarbonate, metal, ceramic, plastic, and another suitable material. In some embodiments, the housing 14 may include a proximal end 16, a distal end 18, and a slot 20. In some embodiments, the slot 20 may extend parallel to a longitudinal axis of the housing 14.

In some embodiments, the instrument delivery device 10 may include an advancement element 22, which may extend through the slot 20 and may be configured to move linearly along the slot 20 between a retracted position illustrated, for example, in FIG. 1A, and an advanced position distal to the retracted position. In some embodiments, the retracted position may correspond to a fully retracted position with the advancement element 22 at a proximal end of the slot 20. In some embodiments, the clinician may pinch or grasp the advancement element 22 to move the advancement element 22 between the retracted position and the advanced position.

In some embodiments, the distal end 18 of the housing 14 may include a distal connector 24. In some embodiments, the distal connector 24 may include opposing lever arms 26a,26b. In some embodiments, distal ends of the opposing lever arms 26a,26b may be configured to move apart from each other in response to pressure applied to proximal ends of the opposing lever arms 26a,26b. In some embodiments, in response to removal of the pressure applied to the proximal ends of the opposing lever arms 26a,26b, the distal ends of the opposing lever arms 26a,26b may move closer to each other and clasp a portion of the catheter assembly, such as a needleless connector, another connector, or a proximal end of a catheter adapter, for example. In some embodiments, the distal connector 24 may include a blunt cannula or male luer configured to insert into the portion of the catheter assembly.

In some embodiments, the distal connector 24 may include any suitable connector. For example, the distal connector 24 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 24 may include one or more bond pockets, which may each be configured to receive an extension tube, which may be part of the catheter assembly or extend between the distal connector 24 and the catheter assembly. In some embodiments, the distal connector 24 may be monolithically formed as a single unit with a body of the housing 14 that includes the slot 20.

In some embodiments, the tubing 12 and/or the guidewire 13 may be replaced with any suitable probe or instrument. In some embodiments, the tubing 12, the guidewire 13, or the suitable probe or instrument may include one or more sensors for patient or device monitoring and may include sensors measuring pressure, temperature, pH, blood chemistry, oxygen saturation, flow rate, or another physiological property.

In some embodiments, the guidewire 13 may include a first end 26 and a second end 28. In some embodiments, the first end 26 of the guidewire 13 may be secured within the housing 14. For example, the first end 26 of the guidewire 13 may be fixed to an inner surface of the housing 14. In some embodiments, in response to movement of the advancement element 22 distally a first distance along the slot 20, the second end 28 of the guidewire 13 may be configured to advance a second distance. In some embodiments, the second distance may be twice the first distance. In these embodiments, the advancement element 22 and the guidewire 13 may have a 1:2 advancement ratio such that for a particular distance the advancement element 22 is moved along the slot 20, the second end 28 of the guidewire 13 is moved twice the particular distance.

In some embodiments, the guidewire 13 may include any suitable shape. For example, the guidewire 13 may include a coil. In some embodiments, the second end 28 of the guidewire may be blunt and/or rounded to prevent damage to vasculature of a patient.

In some embodiments, the tubing 12 may include a distal end 30 and a proximal end 32. In some embodiments, in response to movement of the advancement element 22 distally the first distance along the slot 20, the distal end 30 of the tubing 12 is configured to advance the first distance. In these embodiments, the advancement element 22 and the tubing 12 may have a 1:1 advancement ratio such that for a particular distance the advancement element 22 is moved along the slot 20, the distal end 30 of the tubing 12 is moved a distance equal to the particular distance.

In some embodiments, the proximal end 32 of the tubing 12 may be coupled to the advancement element 22. In some embodiments, the instrument delivery device 10 may include an extension tube 34 coupled to the advancement element 22, and a blood collection pathway 36 may extend through the tubing 12, the advancement element 22, and the extension tube 34.

In some embodiments, the instrument delivery device 10 may include a seal 38 disposed within the advancement element 22 and preventing fluid communication between the blood collection pathway 36 and a portion of a guidewire pathway through which the guidewire 13 moves. In some embodiments, the seal 38 may include an elastomeric septum. In some embodiments, the guidewire 13 may include the coil or another suitable fluid permeable structure. In some embodiments, the coil or the other suitable fluid permeable structure may be distal to the seal 38 or extend through the seal 38 when the advancement element 22 is in an initial or fully retracted position.

In some embodiments, the coil may include multiple loops wound around a central axis and may or may not include a central core wire. In some embodiments, the guidewire 13 may be constructed of metal, stainless steel, nitinol, or another suitable material. In some embodiments, the guidewire 13 and/or the coil may be similar or identical in terms of one or more features and/or operation to the wire and the coil portion, respectively, described further in U.S. Patent Publication No. 2021/0402152, which is hereby incorporated in its entirety.

In some embodiments, in response to movement of the advancement element 22 distally the first distance along the slot 20, the second end 28 of the guidewire 13 and the distal end 30 of the tubing 12 may move from inside the housing 14 to outside of the housing 14. In some embodiments, in response to the advancement element 22 being disposed at a proximal end of the slot 20, the second end 28 of the guidewire 13 and the distal end 30 of the tubing 12 may be aligned with or proximal to a distal end 40 of the blunt cannula or male luer of the distal connector 24, which may protect the guidewire 13 and the tubing 12 and prevent contamination thereof.

In some embodiments, the inner surface 42 of the housing 14 may include one or more grooves, which may be disposed between the proximal end 16 of the housing 14 and the distal end 18 of the housing 14. For example, the inner surface 42 may include a first groove 44 and/or a second groove 46. In some embodiments, the first groove 44 and/or the second groove 46 may be disposed within the housing 14 between the proximal end 16 and the distal end 18. In some embodiments, the tubing 12 may be disposed within the first groove 44, which may provide guidance of the tubing 12. In some embodiments, the guidewire 13 may be disposed within the first groove 44 and the second groove 46, which may provide guidance for the guidewire 13. In some embodiments, the first groove 44 and/or the second groove 46 may include a support wall 48, another support wall 50 opposite the support wall, and a bottom 52 extending between the support wall 48 and the other support wall 50. In some embodiments, the first groove 44 and/or the second groove 46 may be open opposite the bottom 52. In some embodiments, the first groove 44 and/or the second groove 46 may be linear and/or configured to guide the guidewire 13 as the guidewire 13 is advanced distally and/or retracted proximally.

In some embodiments, the guidewire 13 may be disposed in the first groove 44 and/or the second groove. In some embodiments, the tubing 12 may be disposed within the first groove 44. In some embodiments, the first groove 44 and/or the second groove 46 may extend from the distal end 18 towards the proximal end 16 along all or a portion of a path on which the advancement element 22 travels. In some embodiments, the instrument delivery device 10 may include a support feature, which may be configured to contact the tubing 12 to prevent the tubing 12 and/or the guidewire 13 from buckling. Some example support features are further described in U.S. patent application Ser. No. 17/701,124, filed Mar. 22, 2022, and entitled “VASCULAR ACCESS DEVICE TO REDUCE BUCKLING OF AN INSTRUMENT,” which is hereby incorporated by reference in its entirety.

In some embodiments, the advancement element 22 may include an arc-shaped channel 54, which may be U-shaped. In some embodiments, the guidewire 13 may extend and move through the arc-shaped channel 54. In some embodiments, in response to movement of the advancement element 22 a first distance, the second end of the guidewire 13 may be configured to advance distally a second distance that is more than twice the first distance. In these and other embodiments, the guidewire 13 may extend through multiple arc-shapes.

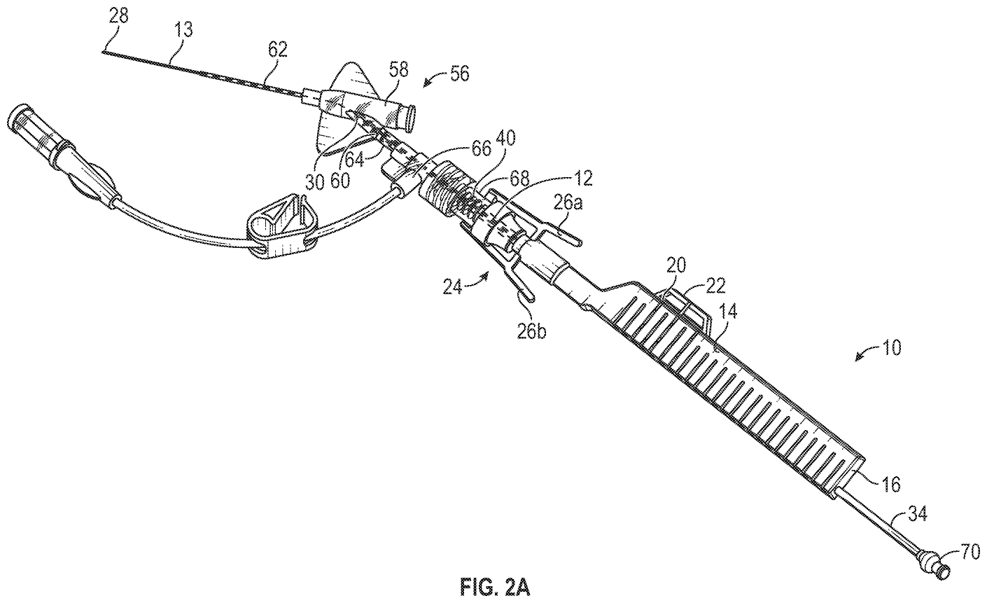

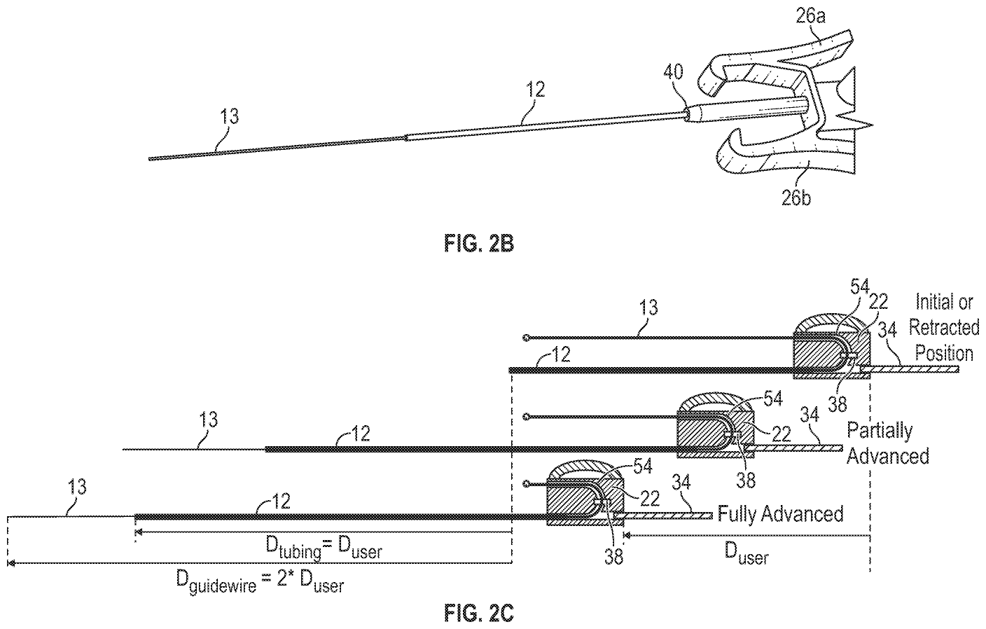

Referring now to FIG. 2A-2B, the advancement element 22, the tubing 12, and the guidewire 13 are illustrated in the fully advanced position, according to some embodiments. In some embodiments, the advancement element 22 may be disposed at a distal end of the slot 20 when the advancement element 22 is in the fully advanced position. In some embodiments, the distal end 30 of the tubing 12 may be disposed within a portion of a catheter assembly 56 when the advancement element 22 is in the fully advanced position.

In some embodiments, the catheter assembly 56 may include a catheter adapter 58, which may include a distal end, a proximal end, and a fluid pathway extending therethrough. In some embodiments, the catheter adapter 58 may include a side port 60 in fluid communication with the fluid pathway of the catheter adapter 58. In some embodiments, the catheter assembly 56 may include a catheter 62, which may be secured within the catheter adapter 58 and may extend distally from the distal end of the catheter adapter 58. In some embodiments, the catheter assembly 56 may be inserted into vasculature of a patient via an introducer needle (not illustrated), which may extend through the catheter 62 and may be used to pierce skin and vasculature of the patient to insert the catheter 62. In some embodiments, the introducer needle may be removed from the catheter assembly 56 after the catheter 62 is placed in the vasculature.

In some embodiments, an extension tube 64 may extend proximally from the side port 60 and/or may be connected to a T-connector 66 or another suitable connector. In some embodiments, the T-connector 66 or the other suitable connector may be connected to a needleless connector 68, which may be connected to the distal connector 24 of the instrument delivery device 10.

In some embodiments, in response to the catheter 62 being inserted into the vasculature, blood may be configured to flow proximally from the catheter 62 through one or more of the catheter adapter 58, the side port 60, the extension tube 64, the T-connector 66, and the needleless connector 68. In some embodiments, the blood may then be configured to flow into the distal connector 24 and proximally through the blood collection pathway 36 of the instrument delivery device 10. In some embodiments, a proximal end of the extension tube 34 may be integrated with a connector 70 which may be coupled to or monolithically formed with a blood collection device, such as, for example, a syringe, a BD VACUTAINER® blood collection tube available from Becton Dickinson & Company of Franklin Lakes, N.J., or another suitable blood collection device. In these and other embodiments, the blood collection device may include a vacuum tube receiver having a needle covered by a protective sheath.

As illustrated in FIG. 2A, in some embodiments, when the advancement element 22 is in the fully advanced position, the distal end 30 of the tubing 12 may be disposed within the side port 60 or elsewhere within the catheter adapter 58. As mentioned, in some embodiments, the tubing 12 may extend through the catheter 62 when the advancement element 22 is in the fully advanced position. In some embodiments, a maximum outer diameter of the tubing 12 may be less than portions of the catheter assembly 56 through which the tubing 12 is configured to extend. In some embodiments, the maximum outer diameter of the tubing 12 may be increased to a maximum size that will still fit through the portions of the catheter assembly 56 through which the tubing 12 is configured to extend. In some embodiments, a maximum outer diameter of the guidewire 13, which may include the coil, may be less than a minimum inner diameter of the tubing 12 to facilitate movement of the guidewire 13 through the tubing 12.

Referring now to FIG. 2C, the advancement element 22 is illustrated an initial or fully retracted position, a partially advanced position, and the fully advanced position, according to some embodiments. In some embodiments, the distal end 30 of the tubing 12 may be generally aligned with the second end 28 of the guidewire 13 when the advancement element 22 is in the initial or fully retracted position. D_{tube} may correspond to a distance the tube moves, Dᵤₛₑᵣ may correspond to a distance the advancement element 22 is moved by the user, and D_{guidewire} may correspond to a distance the guidewire 13. As illustrated in FIG. 2C, in some embodiments, D_{tube} may be equal to Dᵤₛₑᵣ and D_{guidewire} may be twice Dᵤₛₑᵣ when the advancement element 22 is fully advanced by the user.

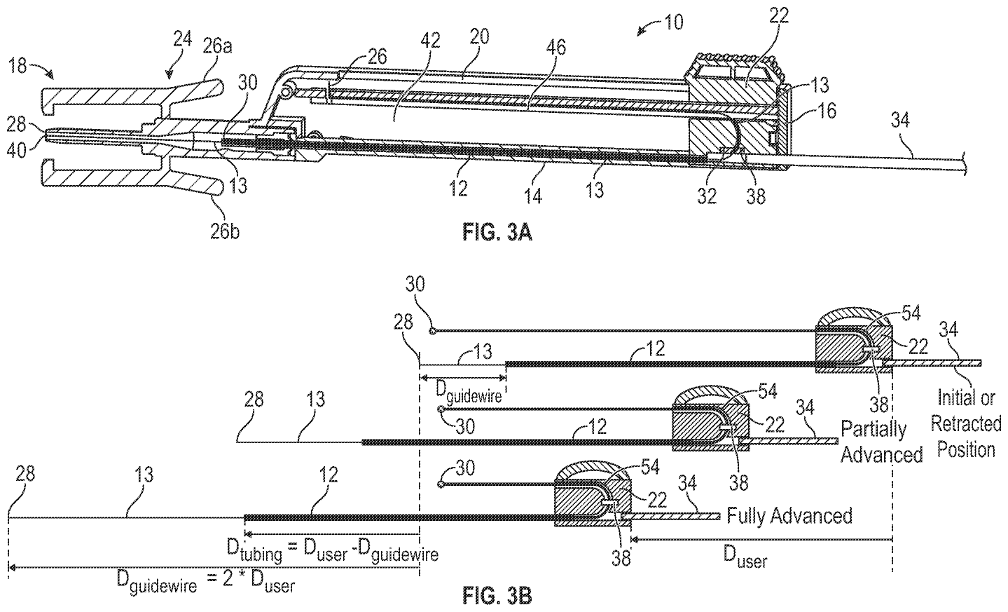

Referring now to FIGS. 3A-3B, in some embodiments, the second end 28 of the guidewire 13 may be disposed at a different position than the distal end 30 of the tubing 12 when the advancement element 22 is in the initial or retracted position. In some embodiments, the guidewire 13 and the tubing 12 may advance at different rates. In further detail, the tubing 12 and the guidewire 13 will advance at a 1:1 and 1:2 advancement ratio with respect to the advancement element 22, respectively. In some embodiments, a length of the tubing 12 may be selected to position the distal end 30 at a desired location within a particular catheter assembly when the advancement element 22 is fully advanced.

In FIG. 3B, D_{tubing} is equal to Dᵤₛₑᵣ, and D_{guidewire} is twice Dᵤₛₑᵣ. In some embodiments, the second end 28 of the guidewire 13 may be disposed at a different position than the distal end 30 of the tubing 12 when the advancement element 22 is in the initial or retracted position, but advancement ratios of the tubing 12 and the guidewire 13 with respect to the advancement element 22 may be the same as in FIG. 2C. In further detail, in some embodiments, the tubing 12 and the guidewire 13 will advance at a 1:1 and 1:2 advancement ratio with respect to the advancement element 22, respectively.

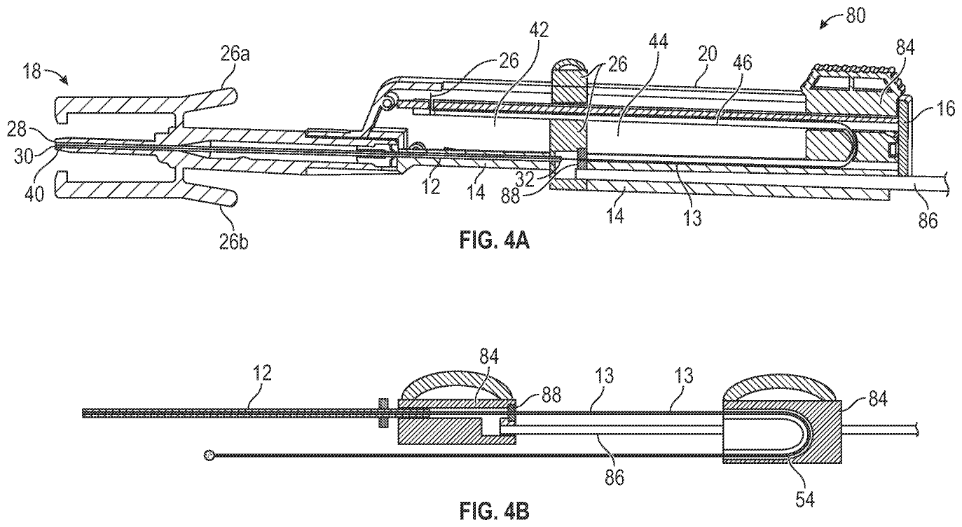

Referring now to FIGS. 4A-4B, an instrument delivery device 80 is illustrated, according to some embodiments. In some embodiments, the instrument delivery device 80 may be similar or identical to the instrument delivery device 10 of FIGS. 1A-3B in terms of one or more features and/or operation. In some embodiments, instrument delivery device 80 may include a first advancement element 82, which may be similar or identical to the advancement element 22 of FIGS. 1A-3B in terms of one or more features and/or operation. In some embodiments, the first advancement element 82 may extend through the slot 20 and configured to move linearly along the slot 20. In some embodiments, the instrument delivery device 80 may include a second advancement element 84 extending through the slot 20 and configured to move linearly along the slot 20.

In some embodiments, the instrument delivery device 80 may include the guidewire 13, which may include the first end 26 and the second end 28. In some embodiments, in response to movement of the first advancement element 82 distally a first distance along the slot 20, the second end of the guidewire 13 may be configured to advance a second distance that is twice the first distance. In these embodiments, the first advancement element 82 and the guidewire 13 may have a 1:2 advancement ratio such that for a particular distance the first advancement element 82 is moved along the slot 20, the second end 28 of the guidewire 13 is moved twice the particular distance.

In some embodiments, the instrument delivery device 80 may include the tubing 12, which may include the distal end 30 and the proximal end 32. In response to movement of the second advancement element 84 distally the first distance along the slot 20, the distal end 30 of the tubing 12 may be configured to advance the first distance. In these embodiments, the second advancement element 84 and the tubing may have a 1:1 advancement ratio such that for a particular distance the second advancement element 84 is moved along the slot 20, the distal end 30 of the tubing 12 is moved the particular distance.

In some embodiments, the proximal end 32 of the tubing 12 may be coupled to the second advancement element 84. In some embodiments, the instrument delivery device 80 may include another tubing 86 coupled to the second advancement element 84. In some embodiments, a blood collection pathway may extend through the tubing 12, the second advancement element 84, and the other tubing 86.

In some embodiments, the instrument delivery device 80 may include a seal 88 disposed within the second advancement element 84 and preventing fluid communication between the blood collection pathway and a portion of a guidewire pathway. In some embodiments, the guidewire 13 may extend through the seal 88, which may include an elastomeric septum.

In some embodiments, the first advancement element 82 may include the arc-shaped channel 54, and the guidewire 13 may move within the arc-shaped channel 54. In some embodiments, the first end 26 of the guidewire 13 may be secured within the housing 14. In some embodiments, the second advancement element 84 may be distal to the first advancement element 82. In some embodiments, in response to movement of the first advancement element 82 distally the first distance along the slot 20, the second end 28 of the guidewire 13 and the distal end 30 of the tubing 12 may move from inside the housing 14 to outside of the housing 14.

In some embodiments, the second advancement element 84 may be advanced distally prior to advancement of the first advancement element 82, and the tubing 12 may create a seal with an inner diameter of a portion of a particular catheter assembly, such as a T-connector. In some embodiments, the first advancement element 82 may then be advanced distally through the tubing 12 and/or beyond the catheter of the particular catheter assembly.

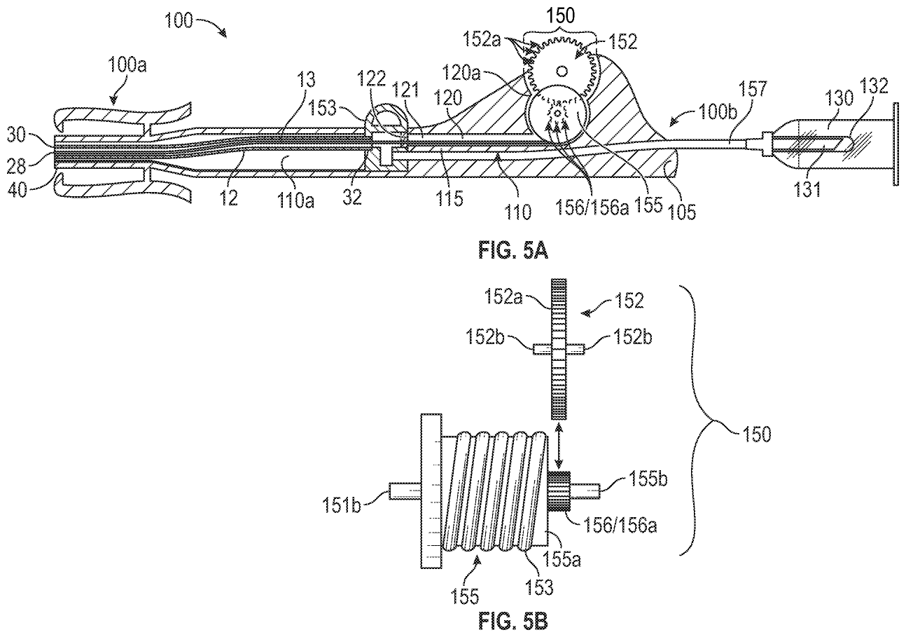

Referring now to FIGS. 5A-5B, an instrument delivery device 100 is illustrated, according to some embodiments. In some embodiments, the instrument delivery device 100 may be similar or identical to the instrument delivery device 10 of FIGS. 1A-3B and/or the instrument delivery device 80 of FIGS. 4A-4B in terms of one or more features and/or operation. In some embodiments, the instrument delivery device 100 may include the housing 14 having the distal end 18 and the proximal end 16. In some embodiments, although only a portion of the distal end 18 is illustrated, the distal end 18 could include any type of connector to enable the instrument delivery device 100 to be connected to a catheter assembly or could incorporate an intravenous catheter. In some embodiments, the proximal end 16 may be configured to form a vacuum tube receiver 130 having a needle 131 covered by a protective sheath 132.

In some embodiments, the blood collection pathway 36 may extend within the instrument delivery device 100 from the needle 131 to the distal end 18. Accordingly, when a vacuum tube is inserted into the vacuum tube receiver 130, a blood sample can be collected through the blood collection pathway 36. In some embodiments, the proximal end 16 may include a Luer connector or any other type of connector that is coupled to the blood collection pathway 36.

In some embodiments, the instrument delivery device 100 may include an instrument delivery mechanism 150 that enable the guidewire 13 to be advanced in a distal direction through a catheter assembly and/or subsequently withdrawn in a proximal direction. In some embodiments, the guidewire 13 may include a wire constructed of nitinol, nickel titanium, or another suitable material. In some embodiments, a compartment 120 may be formed within the instrument delivery device 100 and may house the instrument delivery mechanism 150. In some embodiments, a dividing wall 115 may create an instrument channel 121 that extends distally from the compartment 120 and may be configured to be proximate the seal 38 secured with an advancement element 153. In some embodiments, the seal 38 (e.g., an elastomeric septum) may isolate the compartment 120 from the blood collection pathway 36.

In some embodiments, the guidewire 13 may be configured to move through the seal 38. In some embodiments, the guidewire 13 may extend through a slit or other opening formed within seal 38. In some embodiments, the seal 38 may provide support to the guidewire 13 to prevent it from buckling as it is advanced. Although the instrument channel 121 is illustrated as being substantially wider than guidewire 13, in some embodiments, dimensions of at least a portion of the instrument channel 121 may be only slightly greater than the guidewire 13 so that instrument channel 121 may provide support to prevent buckling of the guidewire 13. In some embodiments, the seal 38 may be configured to prevent blood from flowing into the housing 14.

In some embodiments, the instrument delivery mechanism 150 may include a spool 155 and an advancement wheel 152, both of which may be configured to rotate within the compartment 120. In some embodiments, the spool 155 may be positioned adjacent to the advancement wheel 152 (i.e., towards the instrument channel 121 relative to the advancement wheel 152). In some embodiments, the advancement wheel 152 may be positioned to extend partially out from the compartment 120 to thereby enable the user to use his or her thumb or finger to rotate the advancement wheel 152. In some embodiments, the spool 155 may include a gear 156 having teeth 156a. Likewise, in some embodiments, the advancement wheel 152 may include teeth 152a and may therefore function as a gear. In some embodiments, the teeth 152a may interface with the teeth 156a so that the spool 155 is rotated when the advancement wheel 152 is rotated. In some embodiments, the teeth 152a are formed along the outermost edge of the advancement wheel 152. In other embodiments, however, the teeth 152a may be formed along a portion of the advancement wheel that is inset relative to the outermost edge.

FIG. 5B provides an exploded rear view of the instrument delivery mechanism 150 in isolation, according to some embodiments. In some embodiments, the spool 155 and the advancement wheel 152 may include axles 155b and 152b, respectively, by which these components are positioned within the compartment 120 and around which these components rotate. In some embodiments, the spool 155 may include a spool drum 155a around which the guidewire 13 may be wound. Therefore, when the spool 155 is rotated, the rotation may cause the guidewire 13 to be advanced or retracted along the instrument channel 121 depending on the direction in which the advancement wheel 152 is rotated.

In some embodiments, the gear formed by the advancement wheel 152 may have a larger diameter than the gear 156 to thereby cause the guidewire 13 to be advanced or retracted a larger distance relative to the amount of rotation of the advancement wheel 152. In contrast, in other embodiments, the gear formed by the advancement wheel 152 may have an equal or smaller diameter than the gear 156. In such embodiments, the guidewire 13 may advance or retract a smaller distance relative to the amount of rotation of the advancement wheel 152 but such advancement or retraction may be accomplished with a reduced amount of force to the advancement wheel 152.

In some embodiments, in response to the advancement wheel 152 being rotated, the guidewire 13 may be configured to advance through the advancement element 153, the tubing 12, and the distal end 18 of the housing 14. In some embodiments, the instrument delivery device 100 may include another tubing 157 coupled to the advancement element 153 and configured to couple to the vacuum tube receiver 130 or another blood collection device. In some embodiments, blood may be configured to flow proximally through the tubing 12, the advancement element 153, and the other tubing 157 for collection. In some embodiments, the advancement wheel 152 may include or correspond to any other advancement wheel described in further detail in U.S. patent application Ser. No. 17/709,935, filed Mar. 31, 2022, entitled “INSTRUMENT DELIVERY DEVICES, SYSTEMS, AND METHODS,” which is hereby incorporated in its entirety.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. An instrument delivery device, comprising:
a housing, comprising a proximal end, a distal end, and a slot;
an advancement element extending through the slot and configured to move linearly along the slot;
a guidewire comprising a first end and a second end, wherein in response to movement of the advancement element distally a first distance along the slot, the second end of the guidewire is configured to advance a second distance, wherein the second distance at least twice the first distance; and
a tubing comprising a distal end and a proximal end, wherein in response to movement of the advancement element distally the first distance along the slot, the proximal end of the tubing is configured to advance the first distance.

2. The instrument delivery device of claim 1, wherein the proximal end of the tubing is coupled to the advancement element.

3. The instrument delivery device of claim 1, further comprising an extension tube coupled to the advancement element, wherein a blood collection pathway extends through the tube, the advancement element, and the extension tube.

4. The instrument delivery device of claim 3, further comprising a seal disposed within the advancement element and preventing fluid communication between the blood collection pathway and a portion of a guidewire pathway.

5. The instrument delivery device of claim 1, wherein the advancement element comprises an arc-shaped channel, wherein the guidewire moves within the arc-shaped channel, wherein the first end of the guidewire is secured within the housing.

6. The instrument delivery device of claim 1, wherein in response to movement of the advancement element distally the first distance along the slot, the second end of the guidewire and the distal end of the tubing move from inside the housing to outside of the housing.

7. The instrument delivery device of claim 1, wherein a distal end of the housing comprises a distal connector, wherein in response to the advancement element being disposed at a proximal end of the slot, the second end of the guidewire and the distal end of the tubing are aligned with a distal end of the distal connector.

8. The instrument delivery device of claim 1, wherein a distal end of the housing comprises a distal connector, wherein in response to the advancement element being disposed at a proximal end of the slot, the second end of the guidewire and the distal end of the tubing are proximal to a distal end of the distal connector.

9. The instrument delivery device of claim 1, wherein an inner surface of the housing comprises a groove disposed between the proximal end of the housing and the distal end of the housing, wherein the guidewire and the tubing are disposed within the groove.

10. An instrument delivery device, comprising:
a housing, comprising a proximal end, a distal end, and a slot;
a first advancement element extending through the slot and configured to move linearly along the slot;
a second advancement element extending through the slot and configured to move linearly along the slot;
a guidewire comprising a first end and a second end, wherein in response to movement of the first advancement element distally a first distance along the slot, the second end of the guidewire is configured to advance a second distance, wherein the second distance is at least twice the first distance; and
a tubing comprising a distal end and a proximal end, wherein in response to movement of the second advancement element distally the first distance along the slot, the distal end of the tubing is configured to advance the first distance.

11. The instrument delivery device of claim 10, wherein the proximal end of the tubing is coupled to the second advancement element.

12. The instrument delivery device of claim 10, further comprising another tubing coupled to the second advancement element, wherein a blood collection pathway extends through the tubing, the second advancement element, and the other tubing.

13. The instrument delivery device of claim 10, further comprising a seal disposed within the second advancement element and preventing fluid communication between the blood collection pathway and a portion of a guidewire pathway, wherein the instrument extends through the seal.

14. The instrument delivery device of claim 10, wherein the first advancement element comprises an arc-shaped channel, wherein the guidewire moves within the arc-shaped channel, wherein the first end of the guidewire is secured within the housing.

15. The instrument delivery device of claim 10, wherein the second advancement element is distal to the first advancement element.

16. The instrument delivery device of claim 10, wherein in response to movement of the first advancement element distally the first distance along the slot, the second end of the guidewire and the distal end of the tubing move from inside the housing to outside of the housing.

17. An instrument delivery device, comprising:
a housing, comprising a distal end, a proximal end, and a slot disposed between the distal end and the proximal end, wherein the distal end is configured to couple to an intravenous catheter device;
a tubing, comprising a distal end and a proximal end;
an advancement element through the slot and configured to move linearly along the slot, wherein the advancement element is coupled to the proximal end of the tubing, wherein in response to movement of the advancement element distally a first distance along the slot, the distal end of the tubing is configured to advance the first distance;
an instrument disposed within the housing; and
an advancement wheel, wherein the advancement wheel extends out from the housing, wherein in response to the advancement wheel being rotated, the instrument is configured to advance through the advancement element, the tubing, and the distal end of the housing.

18. The instrument delivery device of claim 17, wherein the instrument comprises a guidewire.

19. The instruments delivery device of claim 17, further comprising a septum disposed within the advancement element and configured to prevent blood from flowing into the housing, wherein the guidewire is configured to move through the septum.

20. The instrument delivery device of claim 17, further comprising another tubing coupled to the advancement element and configured to couple to a blood collection device, wherein blood is configured to flow proximally through the tubing, the advancement element, and the other tubing.
